(19) United States
(12) Patent Application Publication
(10) Pub. No.: US 2021/0341748 A1
(43) Pub. Date: Nov. 4, 2021

(54) Loupe System

(71) Applicant: Bryant Medical Ltd, Chertsey (GB)

(72) Inventors: Priyam PATEL, Surrey (GB); Connor BRYANT, Ottershaw (GB)

(21) Appl. No.: 17/245,780

(22) Filed: Apr. 30, 2021

Related U.S. Application Data
(63) Continuation-in-part of application No. 16/864,167, filed on May 1, 2020.

(51) Int. Cl. G02C 3/00; A61B 90/00

(52) U.S. Cl. CPC G02C 3/003; G02C 2200/02; A61B 90/08

(57) ABSTRACT

A head strap (120) for medical loupe glasses is provided, comprising two tubes (130, 132) of flexible wipe-clean material and a magnet (134, 136) located on each tube, the magnets (134, 136) being connectable by magnetic forces to effect securement.

### CROSS-REFERENCE TO RELATED APPLICATIONS

This application is a continuation-in-part of U.S. patent application Ser. No. 16/864,167 filed May 1, 2020. The entire disclosure of the above application is incorporated by reference.

### FIELD

The present invention relates broadly to vision aids, and more specifically to retaining corrective lenses to the head of a person.

### BACKGROUND

Loupes are small magnification devices used to see small details more closely. As dental professionals use both hands in performing dental procedures, dental loupes are binocular and usually take the form of a pair of glasses.

Loupes are worn by dentists not only to increase the details that can be seen, but also to improve posture by avoiding slouching to view inside a mouth. Many dentists mount a headlight, with an associated battery pack, on the loupes to increase visibility.

Loupes are supplied ‘fitted’ to the dentist's face and can be supplied with prescription lenses for vision correction. Loupes are also common in other professional sectors such as surgery and single eyeglass loupes in professional sectors such as jewellery, geology and printing.

Many dentists use common eyewear head straps, also known as retainers, lanyards or straps, attached to the temples (the arms) of the loupe frames. Dentists attach head straps to prevent the loupes from falling and becoming damaged as well as, in the case of head straps with an adjustable length, securing the loupes more tightly on the face to prevent them slipping down the nose bridge when looking down or becoming unsettled as the dentists moved his head up or down or rotated left/right.

The risk of loupes slipping off a face is much greater than that of wearing normal eyewear as the loupes are heavier because of the magnification lenses and optional headlight, and also because the loupes are unbalanced and have a heavier weight load on the nose pads due to the mounting of the lens and headlight compared to the temples.

Technology is progressing to reduce the weight of loupes, however at the same time technology advancements mean that higher magnification loupes are becoming available with longer and heavier lenses.

In 1942 Corcoran (U.S. Pat. No. 2,270,931) disclosed a method of supporting heavy optical instruments over eyes. By holding the heavy optical instruments using a pair of parallel bars, which are hinge mounted to head gear comprising two or three fixed length, elongated straps pivotally attached to each other and mounted over the head the weight of the instruments is spread over the head. This arrangement also provides a hands-free method to ensure the optical instruments are always positioned over the eyes pointing forwards as the head moves.

Later in 1955 Schmidt (U.S. Pat. No. 2,935,910) disclosed a method to construct a headband loupe for doctors and other workers in which the lenses, lens holders and other parts of the loupe are made removable and interchangeable to change the level of magnification and also accommodate people wearing glasses behind the lenses. The loupe is secured using rivets to an elasticated headband fastened with buckles or snaps around the circumference of the head. The use of buckles or snaps allows the headband to be adjusted to ensure a tight fit, and the use of an adjustable bolt between the headband and the lens holders allows for the loupe to be tilted upwards away from the eyes when not required.

Price (U.S. Pat. No. 4,234,910) in 1978 disclosed a method to construct a head supported illumination device consisting of the mounting of fibre optics from a light generating machine onto the temples of existing glasses for physicians and surgeons. To secure the frame of the glasses, an adjustable elasticated strap is used to secure them to the back of the head. The strap is attached to the temples using a rubber loop attached to the end of the temple and is adjusted using a buckle.

Bastable and Goldberg (U.S. Pat. No. 4,807,987) in 1986 disclosed a method to mount an interchangeable set of loupe lens together with a fibre optic light source for ear, nose and throat surgery. The mounting is attached to an upholstered, adjustable headband around the circumference of the head together with an additional strap over the top of the head to allow comfortable use for a number of hours and allow adjustments for individual head sizes using fasteners as well as brackets to position the viewing system and light system in front of the user's eyes.

Later in 1994 Sherlock (U.S. Pat. No. 5,548,841) disclosed a method to construct a headband for a loupe using a one-piece polypropylene headband that provides an easy method to quickly slip the loupes on and off the head, together with a compression force that provides a method to secure the loupe for all head sizes.

Qingfeng (China Patent CN2215728Y) in 1994 disclosed a method to mount magnification lenses with variable magnification on a frame attached to the head using an adjustable rubber belt. This has the benefit of being smaller than traditional loupes and allowing variable magnification. The rubber belt is simple to adjust.

Donny's application in 2019 (WIPO Patent Application PCT/US2019013777) disclosed a method to build a head strap for a standard pair of loupes available for sale from various manufacturers using two monofilaments which are attached to the temples of the loupes using an elasticated coupler. The monofilaments pass through a tight sleeve near each of the temple, with a stopper made of rubber or plastic on the other end. The sleeve allows for size adjustment and the monofilament loop from each temple passes over the back of the head. The method provides an adjustable head strap for existing loupes which is very light weight and displaces the weight of the loupes to the back of the head. The use of monofilaments makes the loop more hygienic and easier to clean.

While these examples provide methods to construct a loupe and headband combinations and also methods to manufacture a headband to attach a standard pair of loupes to a head to displace the weight of the loupes over the head, and to securely attach the magnification lenses so that they remain stable as the head moves, what makes the method in the present application unique from Corcoran, Schmidt, Price, Bastable, Sherlock, Qingfeng and Donny is a method of constructing a head strap for a standard paid of loupes which is hygienic and very easy to disinfect, whilst at the same time being easy and quick to adjust and securely attach to the head, and quickly and securely attach to the temples of a standard loupe. There are no parts which can catch hair or be caught on clothes and the whole surface of the head strap can be simply disinfected using wipes. There is no requirement to adjust for size every time the loupe is placed on the head and the one size head strap is constructed to fit most of the dental and surgical standard loupes available for sale.

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

Aspects and embodiments of the present invention are set out in the appended claims. These and other aspects and embodiments of the invention are also described herein.

According to a first aspect of the invention, there is provided a head strap for medical loupe glasses, comprising two tubes of flexible wipe-clean material and a magnet located on each tube, the magnets being connectable by magnetic forces to effect securement.

This provides a secure and hygienic head strap for medical loupe glasses.

In a preferable implementation, the head strap has a smooth outer surface, preferably wherein the flexible wipe-clean material is silicone.

Preferably, the magnets are moveable. The magnets may be located on toggles slidable over the tubes.

The head strap may further comprise a connector block for connecting the two tubes, preferably wherein the weight of the connector block is adjustable. The connector block can act as a counterbalance, and by choosing different weights of connector block, different loupe arrangements can be balanced.

Preferably, the connector block comprises two connectable parts, preferably wherein the two parts are removably connectable. This can allow the block to be disconnect and removed, reconnected and disconnected, and so on.

The two parts comprise corresponding pegs and holes to facilitate connection.

Preferably, the head strap further comprises a stopper on the end of each tube, configured to anchor the tubes in the connector block.

In a preferred implementation, the toggles, connector block and stoppers can be disconnected from the tubes and subsequently reconnected. They can then be disconnected and reconnected again and so on. This can allow the elements to be cleaned separately between uses, and for elements to be replaced.

Preferably, the stoppers have flared ends. More preferably, the connector block comprises cavities configured to accommodate and anchor the flared ends of the stoppers.

The magnets may have flat faces, preferably the magnets have disc-shaped faces. More preferably, the magnets are located on an external face of the toggles, that face being parallel to the tube.

In a preferably implementation, the toggles comprise a hole configured to accommodate the tube, and a dimension of the toggle parallel to the tube is reduced in the region of the hole. This can allow the tubes to bend relative to the toggle as they pass through the toggle, which can be more comfortable for the wearer. Preferably, the toggles have a generally cylindrical shape. More preferably, the magnets are located at an end of the cylindrical toggles.

Preferably, the tubes are configured to attach to temple arms of loupes and comprise a textured internal surface to engage with temple arms of loupes.

According to a further aspect of the invention, there is provided a loupe system comprising: loupes; and a head strap, comprising two tubes of flexible wipe-clean material and a magnet located on each tube, the magnets being connectable by magnetic forces to effect securement.

Preferably, the magnets are located on toggles slidable over the tubes.

The loupes may comprise temple arms having curved and broadening ends, and the head strap may further comprise a textured internal surface configured to engage the temple arms.

Preferably, the loupes comprise one or more magnification lenses, and the head strap further comprises a connector block, wherein the weight of the connector block is configured to counterbalance the one or more magnification lenses.

In some implementations, the loupes may comprise one or more magnification lenses and a head light, and the head strap may further comprise a connector block, wherein the weight of the connector block is configured to counterbalance the combined weight of the one or more magnification lenses and the head light.

According to a further aspect of the invention, there is provided a loupe system comprising: a loupe comprising: loupe magnification lenses; and temple arms having curved and broadening ends; a head strap formed of flexible material and comprising: at least two magnets; a textured internal surface; and a connector block; wherein the curved and broadening ends of the temple arms and the textured internal surface of the head strap are configured to engage to form a secure connection between the loupe and the head strap; the at least two magnets are connectable by magnetic forces; and the connector block and loupe magnification lenses are configured to act as counterbalances to each other.

The invention can provide an improved loupe system, which is more balanced, comfortable and hygienic, and can be quickly and securely attached and detached.

The head strap may comprise two tubes. The two tubes may preferably be connected by the connector block. The loupe may further comprise a nose bridge, preferably wherein the nose bridge connects the loupe magnification lenses.

In a preferable implementation, the weight of the connector block can be adjusted. The connector block may be removable and replaceable with a connector block of an alternative weight. This can allow the weight of the loupes to be balanced front to back.

The loupe may further comprise a headlight, wherein the connector block has a first weight and the loupe magnification lenses and headlight have a second weight such that the first weight and second weight are configured to act as counterbalances.

According to another aspect of the invention, there is provided a loupe system comprising: a loupe comprising: loupe magnification lenses; a headlight; and temple arms having curved and broadening ends; a head strap formed of flexible material and comprising: at least two magnets; a textured internal surface; and a connector block; wherein the curved and broadening ends of the temple arms and the textured internal surface of the head strap are configured to engage to form a secure connection between the loupe and the head strap; the at least two magnets are connectable by magnetic forces; and the connector block has a first weight and the loupe magnification lenses and headlight have a second weight such that the first weight and second weight are configured to act as counterbalances to each other.

Headlights (and associated battery packs) are often used by dentists, which can be heavy and increase the risk of the loupes falling off; this arrangement can allow the weight of the loupes to be balanced front to back.

In a preferable implementation, the headlight is reversibly attachable, and the connector block is reversibly attachable such that the weight of the connector block can be adjusted. This can ensure a more stable arrangement.

Preferably, the flexible material of the head strap and the curved and broadening ends of the temple arms are configured such that the flexible material stretches to accommodate the curved and broadening ends of the temple arms, and the textured internal surface grips the temple arms, thereby securing connection.

Preferably, the textured internal surface comprises raised surface elements, and the flexible material stretches between the raised surface elements to accommodate the curved and broadening ends of the temple arms. The raised surface elements grips the surface of the temple arms, while allowing the flexible material to stretch between the raised surface elements.

Preferably, the position of the at least two magnets is adjustable. The size of the head strap can be adjusted by moving the magnets.

Preferably, the magnets have flat faces. More preferably, the magnets have disc-shaped faces.

The magnets may be located inside the head strap. Alternatively, the magnet may be located on toggles slidable over the head strap. This can allow the magnets to be moved easily, and the size of the head strap to be thus easily altered. The toggles may be generally cylindrical, and the magnets located on the ends. This can help to prevent hair or clothing becoming tangled. The toggles may be configured such that the faces of the magnets are parallel to the head strap. This facilitates connection of the magnets parallel to the head strap. The toggles may comprise a hole configured to accommodate the head strap, and a dimension of the toggle parallel to the head strap is reduced in the region of the hole. This can allow the head strap to bend around the toggle.

Preferably, the connector block comprises two connectable parts. Preferably the parts are removably connectable. This means the parts can be connected and disconnected, and then reconnected and disconnected and so on. This can allow the head strap to be placed within the connector block, and then the connector block fastened. Preferably the two parts comprise corresponding pegs and holes to facilitate connection. Preferably, the head strap further comprises stopper ends configured to anchor the head strap in the connector block. This provides a secure and reversible connection of the head strap. The magnets and connector block can also be removed for cleaning of the head strap if so desired.

The loupe may be fitted to the face of the user. The magnification lenses may provide vision correction tailored to the user. For example, they may be supplied with prescription lenses.

In a preferred implementation, the head strap has a smooth outer surface. Even more preferably the flexible material is wipe-clean. In a preferred implementation, the flexible material may be silicone. These features assist in making the head strap easy to clean.

According to a further aspect of the invention, there is provided a head strap for medical loupe glasses, comprising two tubes of flexible wipe-clean material and a magnet located on each tube, the magnets being connectable by magnetic forces to effect securement.

This can provide a head strap which can be easily attached and detached, and which is hygienic as it can be easily cleaned.

One or more magnets may be located on each tube. The magnets may be located internally or externally on each tube.

In a preferred implementation, the head strap has a smooth outer surface. Preferably, the flexible wipe-clean material is silicone. This can allow the strap to be cleaned easily.

In a preferable implementation, the magnets are moveable. This can allow the size of the strap to be adjusted.

Preferably, the magnets have flat faces. More preferably, the magnets have disc-shaped faces.

The magnets may be located external to the head strap. In a preferable implementation, the magnets are located on toggles slidable over the head strap. This can allow the magnets to be moved easily, and as such the size of the head strap to be adjusted easily. Preferably, the magnets are located on an external face of the toggles, that face being parallel to the tube. The toggles may be generally cylindrical, and the magnets located on the ends. This can help to prevent hair or clothing becoming tangled. The toggles may be configured such that the faces of the magnets are parallel to the tubes. This facilitates connection of the magnets parallel to the head strap. The toggles may comprise a hole configured to accommodate the tube, and a dimension of the toggle parallel to the tube is reduced in the region of the hole. This can allow the head strap to bend around the toggle.

Preferably, the two tubes have an internal diameter of 4 mm.

In an alternative arrangement, the two tubes have an internal diameter, and the disc-shaped magnets may sit within the tubing and have a diameter, wherein the diameter of the disc-shaped magnets is twice the internal diameter. More preferably, the disc-shaped magnets have a width, and the width of the disc-shaped magnets is 125% of the internal diameter. The internal diameter of the two tubes may be 4 mm, and the diameter of the disc-shaped magnets is 8 mm. More preferably, the width is 5 mm.

Preferably, the head strap further comprises a connector block for connecting the two tubes. The connector block may be detachable and replaceable. The connector block is preferably weighted. Even more preferably, the weight of the connector block is adjustable. This can provide a counterbalance for loupes secured by the head strap.

The connector block may comprise two connectable parts. Preferably the parts are removably connectable. This means the parts can be connected and disconnected, and then reconnected and disconnected and so on. This can allow the connector block to be disassembled and detached from the head strap. Preferably, the two parts comprise corresponding pegs and holes to facilitate connection.

The head may further comprise a stopper on the end of each tube, configured to anchor the tube in the connector block. Preferably the stoppers have flared ends. The stopper can be placed within the connector block, which is then assembled to connect the tubes of the head strap together. This process can be reversed to disconnect the tubes of the head strap. The head strap arrangement may be disassembled for cleaning. The stoppers, connector block and magnets may be removed for cleaning of the tubes and/or each of the components. Preferably, the connector block comprises cavities configured to accommodate and anchor the stoppers. The cavities may be configured to accommodate the flared ends.

Preferably, the toggles, connector block and stoppers can be disconnected from the tubes and subsequently reconnected. This can allow them to be disassembled for cleaning, then reassembled, disassembled and reassembled and so on.

Preferably, the tubes are configured to attach to temple arms of loupes, and comprise a textured internal surface to engage with temple arms of loupes. Preferably, the textured internal surface comprises raised surface elements, and the flexible material stretches between the raised surface elements to accommodate temple arms.

According to a further aspect of the invention, there is provided a loupe apparatus comprising: loupe magnification lenses; temple arms having curved and broadening ends; a head strap attachable to the temple arms, the head strap being formed of a flexible wipe-clean material and having a textured internal surface; wherein the curved and broadening ends of the temple arms engage with the head strap such that the flexible wipe-clean material stretches to accommodate and secure the temple arms, and the textured internal surface grips the temple arms.

According to a yet further aspect of the invention, there is provided a loupe apparatus comprising: loupes of a first weight; head straps connected to the loupes; and a connector block of a second weight connecting the head straps; wherein the first weight and the second weight are selected to counterbalance one another.

In a preferable implementation, the head straps may comprise magnets for securement, preferably reversible securement. Preferably the loupes comprise loupe magnification lenses and a nose bridge connecting the loupe magnification lenses, wherein preferably the nose bridge is configured to balance on a wearer's nose. The apparatus may further comprise temple arms to which the head straps are attachable. Preferably, the temple arms are configured to balance on a wearer's ears. Preferably the first and the second weight are selected such that the loupe system remains stable on the wearer's nose and ears.

According to a further aspect of the invention, there is provided a head strap for medical loupe glasses, comprising: a pair of tubes of flexible material joined together at one end of each tube; and magnets physically attached to each tube at points along its length, wherein said tubes are attachable to temples of a pair of eyeglasses and said magnets are joined together by magnetic attraction.

Preferably the flexible material is silicone, rubber, polyvinyl chloride, polyurethane, nylon, cotton, braided fiberglass, or fluoropolymer.

The tubes may preferably be connected by a weighted block selected from a plurality of blocks having different weights.

Preferably, the tube has a rough inner surface.

According to a yet further aspect of the invention, there is provided a head strap for medical loupe glasses, comprising: a pair of tubes of flexible material joined together at one end of each tube; and magnets inserted in each tube wherein the magnets are free to move within the tubes, wherein said tubes are attachable to temples of a pair of eyeglasses and said magnets are joined together by magnetic attraction.

The flexible material may preferably be silicone, rubber, polyvinyl chloride, polyurethane, nylon, cotton, braided fiberglass, or fluoropolymer.

Preferably, the tubes are connected by a weighted block, the block selected from a plurality of blocks having different weights.

The tube may have a rough inner surface.

The present invention enables a dentist or surgeon to quickly and securely attach a set of loupes to their head whilst ensuring that the loupe head strap is hygienic and very easy to clean, is easy to adjust its size to ensure a tight fit whilst also being easy and quick to attach and detach. The method also ensures that one size of head strap is able to fit many different manufacturers loupes using a safe and hygienic method of attaching them to the loupes.

In one embodiment the dentist or surgeon attaches a head strap made from pieces of silicone tube to the loupes by simply sliding the ends of the two hanging ends over the ends of the loupe temples. The size, and the texture of the inside surface of the silicone ensures a tight fit on most standard loupes whilst the smooth outer surface ensures a highly hygienic surface which is easy to disinfect with wipes. The size of the head band is adjusted by sliding cylindrical magnets located inside the silicone tubes up, or down. Once the loupes are placed on the head, a simple hand movement to touch the two magnets together secures the head strap to the lower back of the head and ensures that the loupes are steady, as well as transferring the weight of the loupes away from the nose bridge to the back of the head. The weight of the connector at the end of the head strap behind the head can be adjusted to balance the weight of the loupes front to back. In this embodiment the head strap is easily disinfected, is light weight, is easily adjusted, fits most manufactured loupes and is simple and quick to attach and detach from the head.

The present invention attaches to the loupes with a simple hand motion and also simply adjusts the size of the head strap while ensuring that the whole assembly is hygienic and easy to disinfect, and can be securely attached to most of the manufactured loupes available today.

Many features and advantages of the present invention will become apparent from reading the following description, when considered in conjunction with the accompanying drawings.

The invention also encompasses a kit of parts for constructing any of the apparatuses or apparatus elements herein described.

The invention extends to methods, system and apparatus substantially as herein described and/or as illustrated with reference to the accompanying figures.

The invention extends to any novel aspects or features described and/or illustrated herein.

Any apparatus feature as described herein may also be provided as a method feature, and vice versa. As used herein, means plus function features may be expressed alternatively in terms of their corresponding structure, such as a suitably programmed processor and associated memory, for example.

Any feature in one aspect of the invention may be applied to other aspects of the invention, in any appropriate combination. In particular, method aspects may be applied to apparatus aspects, and vice versa. Furthermore, any, some and/or all features in one aspect can be applied to any, some and/or all features in any other aspect, in any appropriate combination. It should also be appreciated that particular combinations of the various features described and defined in any aspects of the invention can be implemented and/or supplied and/or used independently.

It should also be appreciated that particular combinations of the various features described and defined in any aspects of the invention can be implemented and/or supplied and/or used independently.

In this specification the word ‘or’ can be interpreted in the exclusive or inclusive sense unless stated otherwise.

Further areas of applicability of the present disclosure will become apparent from the detailed description, the claims, and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings.

One or more aspects will now be described, by way of example only and with reference to the accompanying drawings having like-reference numerals, in which:

FIG. 1 is a side elevation view of a person wearing a pair of loupes with a head strap, in accordance with a first embodiment of the present invention;

FIG. 2 is a perspective view of a person wearing a pair of loupes with a head strap in accordance with the first embodiment of the present invention;

FIG. 3 is a perspective view of a head strap of the first embodiment with the magnets not connected;

FIG. 4 is a perspective view of a head strap of the first embodiment with the magnets connected;

FIG. 5 is a perspective view of the end of the silicone tube of the head strap that is pushed over the temples of the loupes;

FIG. 6 is a side view of a head strap according to a second embodiment of the present invention with the magnets not connected;

FIG. 7 is a side view of a head strap according to the second embodiment with the magnets connected;

FIG. 8a is an exploded perspective view of the magnetic toggle of the second embodiment;

FIG. 8b is an end view of the magnetic toggle;

FIG. 8c is a top view of the magnetic toggle;

FIG. 8d is a cross-sectional side view of the magnetic toggle through the line A-A as shown in FIG. 8b;

FIG. 8e is a through-view of the magnetic toggle;

FIG. 9 is an expanded view of the end of the head strap and the connector block, including a magnified view in the vicinity of connector block;

FIG. 10 is a perspective view of a disconnected connector block within which stoppers are located;

FIG. 11a is a side view of the outer side of one half of the connector block;

FIG. 11b is a perspective view of one half of the connector block;

FIG. 11c is a side view of the inner side of one half of the connector block;

FIG. 11d is a top view of one half of the connector block; and

FIG. 11e is an end view of one half of the connector block.

In the drawings, reference numbers may be reused to identify similar and/or identical elements.

### DETAILED DESCRIPTION

In the preferred embodiment the dentist or surgeon disinfects their head strap by wiping with a disinfect wipe. The user then attaches the head strap to a pair of loupes which the dentist of surgeon owns, purchased from one of the many manufacturers selling such items. The user then adjusts the fitting of the head strap by moving the magnets inside the head strap silicone tubes. Once adjusted the user places the loupes on the head, with the loupes resting on the bridge of the nose and the loupe temples resting over the ears of the user. The head strap is passed over the head and using a simple hand motion the head strap is tightened by pushing the magnets together. The user is then free, with two hands, to continue with his work.

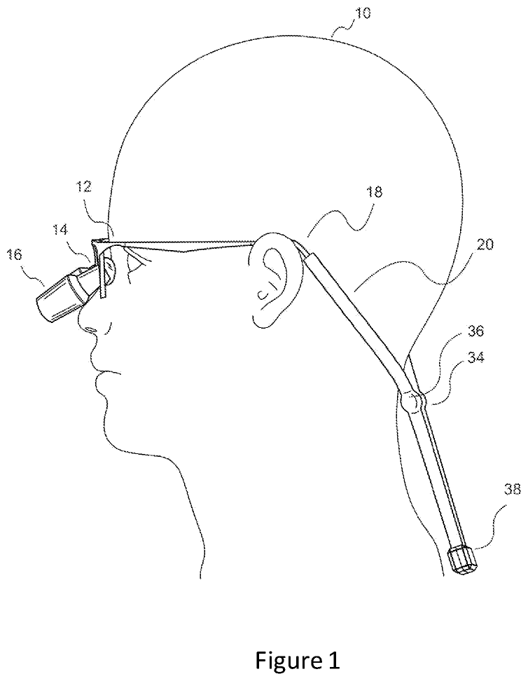

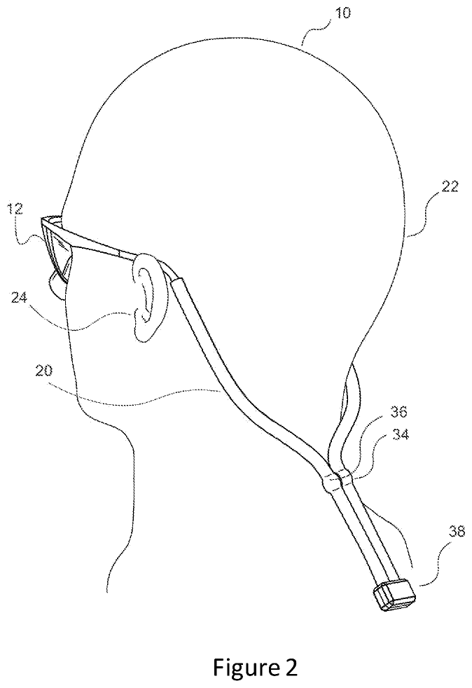

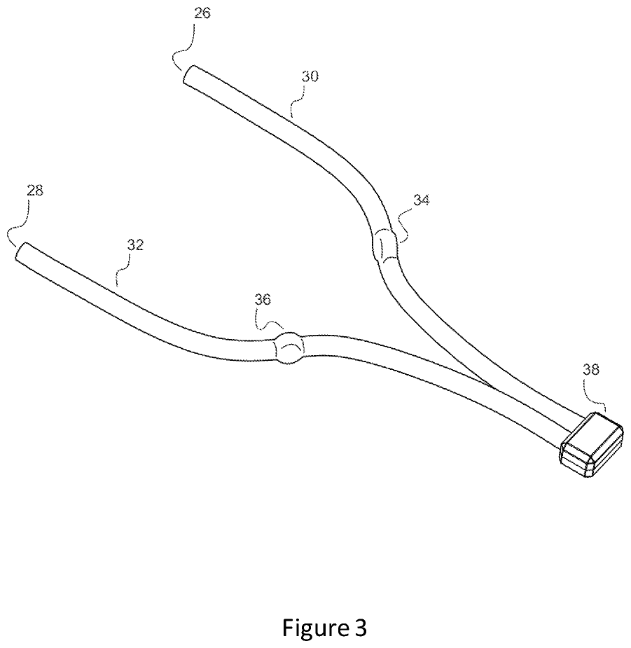

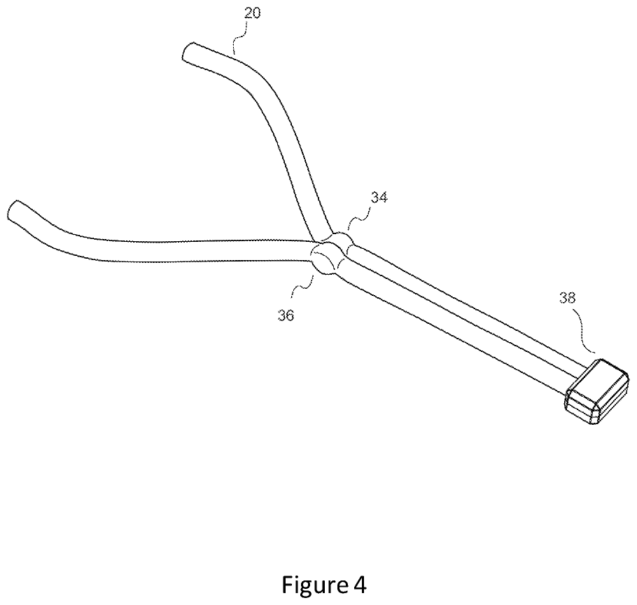

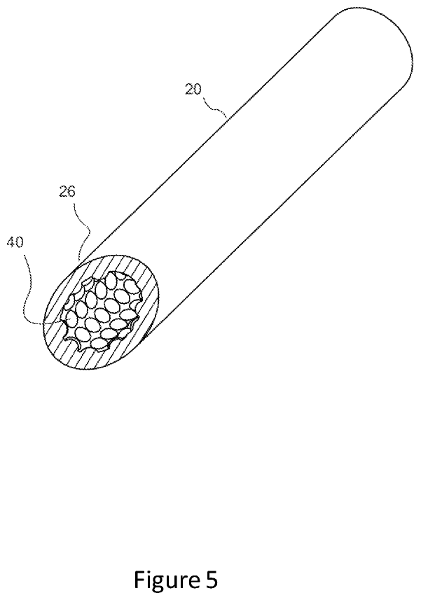

Reference is made to FIG. 1 which shows a side elevation view of a person wearing a pair of loupes with a head strap, FIG. 2 which shows a perspective view of the same person wearing the pair of loupes and head strap, FIG. 3 and FIG. 4, which show perspective views of the head strap on its own and FIG. 5 which shows a perspective view of the end of the silicone tube of the head strap which is attached to the temples of the loupes.

In step 1, the dentist or surgeon disinfects the head strap 20 by wiping the outer smooth silicone surface 30 and 32 and aluminum connector block 38 with a readily available disinfectant wipe, or by boiling the head strap 20 in water for 3 to 4 minutes. The head strap is easy to clean because the uniform construction of the head strap means there are no sharp edges to catch either the wipe or other foreign objects such as hair and also because the head strap is made from silicone material. Silicone is quick-drying, non-porous and hypoallergenic and is 35 times more hygienic than nylon and therefore the head strap is highly more hygienic than a pair of nylon or cotton retainers.

In step 2, if this is the first time that the user is wearing the head strap they will adjust the size of the head strap by moving the Neodymium N52 magnets 34 and 36 located inside the silicone tubes up or down by a gentle squeeze to ensure the distance between the ends of the head strap 26 and 28 and the magnets 36 and 38 is approximately the distance between the ears 22 and 24 and the back of the head.

The disc shaped magnets 36 and 38 have been designed with dimensions of 8 mm diameter and 5 mm width, which sit inside the silicon tubing 30 and 32 which has an internal diameter of 4 mm. This combination of sizes has been found to be optimal to allow the magnets to move without slipping. The magnets 34 and 36 have flat faces to ensure there are no ‘pinch points’ which can wear excessively when the magnets are constantly connected and reconnected.

In step 3, the user attaches the ends of the head strap 26 and 28 to the loupes 12 by inserting the ends of the temples 18 into the ends of the head strap 26 and 28 using a push and twisting motion until tight. The inside of the ends of the silicone tubing 26 and 28 have been designed with a textured internal surface 40 so that the silicone both grips more to the surface of the temples arms using the raised surface elements and also at the same time allows for the silicone material to stretch between the raised surface elements to accommodate as many different sizes and styles of loupe temple arms as possible.

Once the head strap 20 is attached to the loupes 12, in step 4, the user guides the temples of the loupes 18 either side of the head 10 and over the ears 22 and 24 and the nose bridge of the loupes 14 sit on the user's nose Whilst guiding the loupes the user ensures that the head strap 20 is placed over the head 10.

In step 5, once the loupes are comfortable on the ears 22 and 24 and nose 14, the user grasps the magnets 34 and 36 and pushes them together behind the head until the magnets come together due to their magnetic attraction. If the head strap is found to be too loose or tight on their first use, the user can pull the magnets 34 and 36 apart and slide them up or down further inside the silicone tube 30 and 32 as required to readjust the size and then ‘click’ them together again.

During the dental or surgical procedure, the user can view in detail the area to be examined using the loupe magnification lenses 16. If the user needs to move their head from side to side, or up and down, the loupe strap 20 ensures that the loupes 12 remain stable on the nose and ears 22 and 24 and do not slip so much that the view down the magnification lenses 16 is not straight or distorted, or that the loupes 12 do not fall due to their weight and unbalanced nature and potentially break.

In an alternative embodiment, where heavier or larger loupes 12 are required, the aluminum connector block 38 can be replaced with an alternative heavier weighted block to counterbalance the weight of the loupes on the nose, and over the ears 22 and 24 to ensure a more stable sitting of the loupes 12 on the head 10.

Once the user has completed their dental or surgical procedure, the user can pull the magnets 34 and 36 apart to loosen the loupes, and the loupes can be taken on the face in a forward motion, with the head strap 20 passing over the head 10.

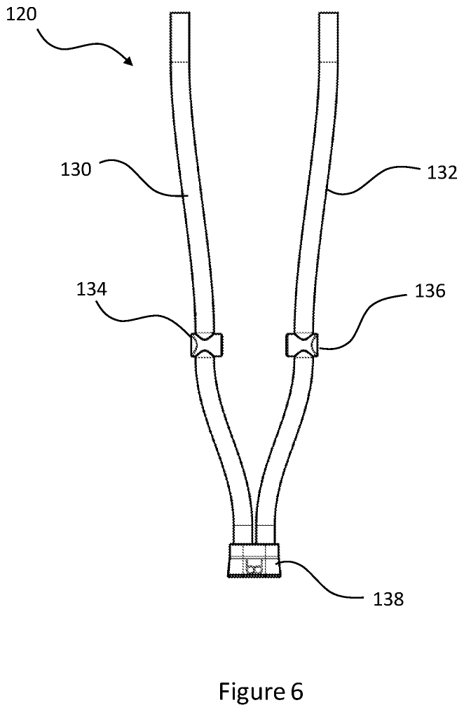

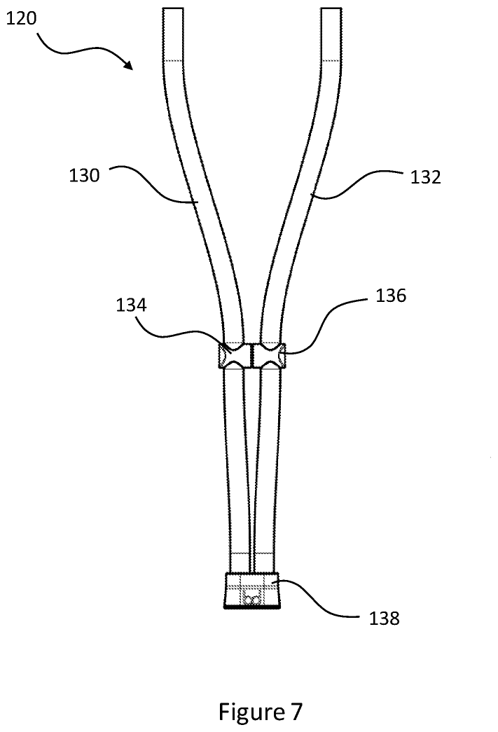

In the first embodiment, as illustrated in FIGS. 1 to 4, the magnets 34 and 36 are located inside the flexible tubing of the loupe straps 30 and 32. FIGS. 6 to 9 illustrate a further embodiment in which a toggle arrangement is used, such that the magnets are positioned on the outer surface of the tubing. FIG. 6 shows the head strap 120 of this second embodiment in an unconnected position. A magnetic toggle 134 and 136 sits over each of the flexible tubes 130 and 132, and these tubes are connected by the connector block 138. FIG. 7 shows this same arrangement in a connected position. The head strap can be positioned over the user's head and connected using the same motions as previously described.

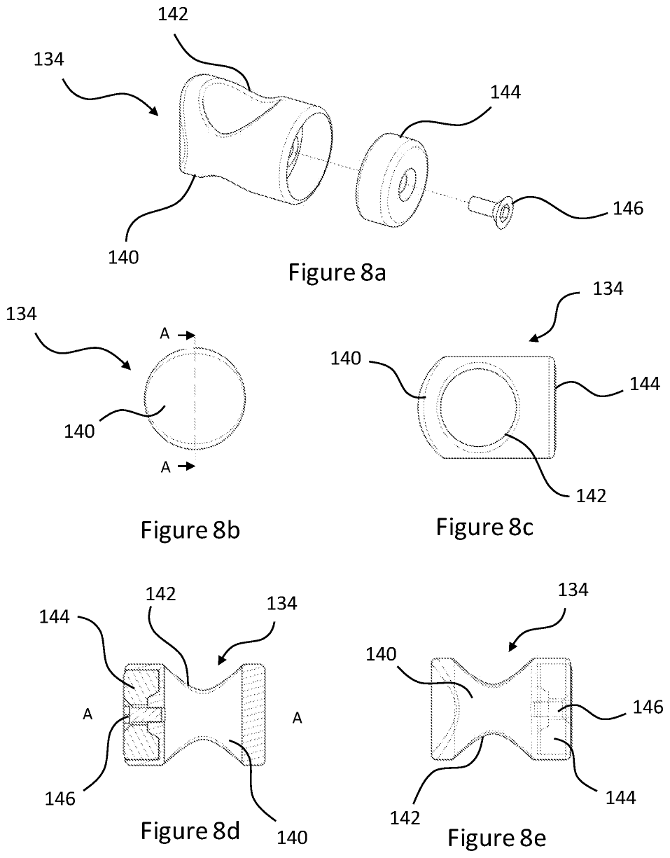

FIG. 8a shows an exploded view of the magnetic toggle 134, which consists of a generally cylindrical body 140 having a hole 142 through which the tubes of the head strap can pass. The toggle 134 further consists of a magnet 144, which can be attached to the body 140 via a screw 146. The magnet 144 is generally disc-shaped and sits at one end (or face) of the cylindrically shaped toggle 134. The hole 142 passes through the longitudinal sides of the toggle 134, such that the tubes of the head strap can pass parallel to the flat face of the disc-shaped magnet 144.

FIG. 8b shows an end view of the toggle 134, where the body 140 has a gently curved end, and FIG. 8c shows a side view of the toggle 134 including the hole 142. FIG. 8d shows a cross-sectional view along the line A-A as indicated in FIG. 8b. A screw 146 passes through the centre of the face of the disc of the magnet 144 through to the body 140 of the toggle 134, and thereby retains the magnet in place. In the vicinity of the hole 142, the diameter of the cylindrically shaped toggle 134 is reduced; this can facilitate bending of the tube as it passes through the hole 142. The flat face of the magnet 144 in the toggle 134 will face the flat face of a magnet in the other toggle 136, and the toggles 134 and 136 can thus be connected by the magnetic forces between them, as illustrated in FIG. 7. The configuration of connection between two flat end faces of a generally cylindrical toggle minimizes the number of potential pinch points, which helps to prevent hair or clothing becoming caught or tangled. The toggles 134 and 136 can be disconnected easily by pulling the magnets apart. The toggles 134 and 136 can be easily moved along the tubes 130 and 132 passing through the holes 142, by which movement the size of the head strap can be adjusted to fit the user.

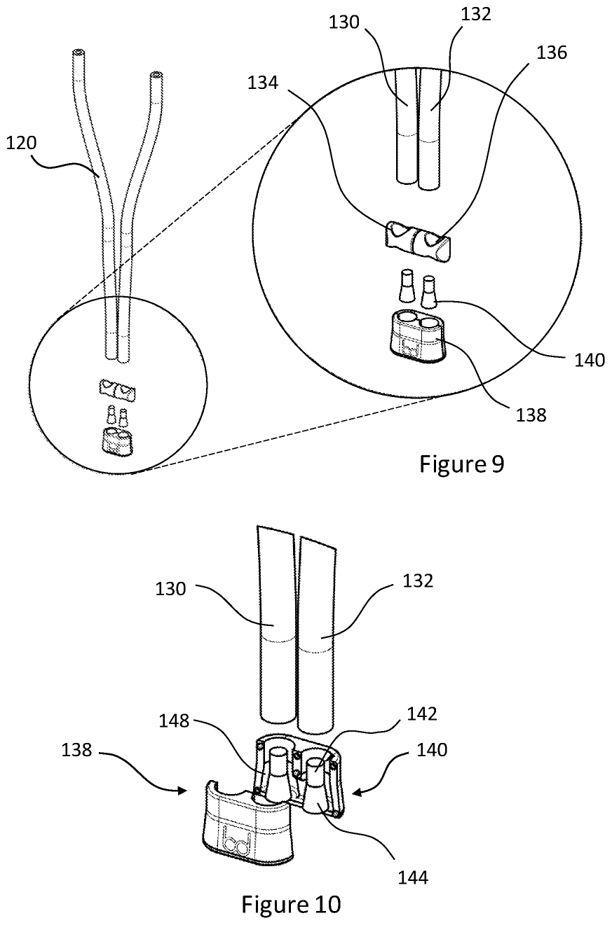

FIG. 9 provides an exploded view of the ends of the tubes 130 and 132, the toggles 134 and 136, stoppers 140, and the connector block 138. FIG. 10 shows an arrangement where the stoppers are positioned within the connector block 138. The toggles 134 and 136 can be threaded onto the tubes 130 and 132. The stoppers 140 can be fitted to the ends of the tubes 130 and 132, preferably by inserting the neck 142 of each stopper 140 within the end of each of the tubes 130 and 132. The tubes stretch and grip the stopper necks 142 to hold the stoppers 140 in place. This end of the tubes 130 and 132 may also be textured to facilitate this. The stoppers have flared ends 144, which extend out to a diameter wider than the diameter of the tubes 130 and 132. As can be seen in FIG. 10, the flared ends 144 of the stoppers 140 engage with correspondingly shaped cavities 148 within the connector block 138. The neck portion of the cavities 148 of the connector block 138 have a diameter larger than the outer diameter of the tubes 130 and 132, but smaller than the outer diameter of the flared ends 144 of the stoppers 140. When the stoppers 140 are attached to the ends of the tubes 130 and 132, the ends of the tubes and stoppers 140 can be placed within one half of the connector block 138. Once the two halves of the connector block 138 are joined, the flared ends 144 are clamped by the two halves to anchor the stoppers 140 within the connector block 138, thereby connecting the ends of the tubes 130 and 132.

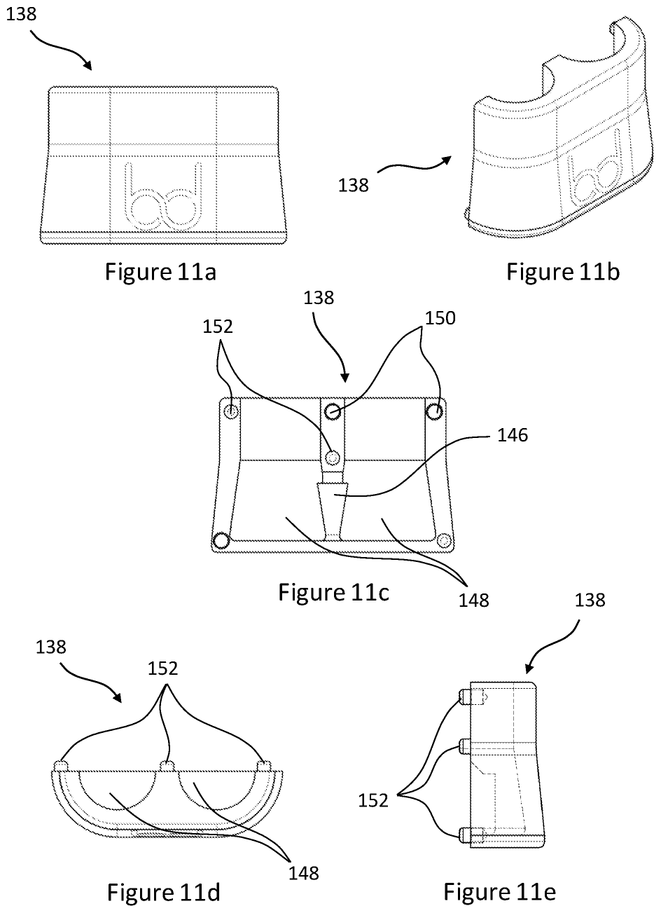

FIGS. 11a to 11e show different views of one half of the connector block 138. In particular, FIG. 11c shows that there is a flared post 146 between the two cavities 148 that accommodate the flared ends 144 of the stoppers 140. The flared post 146 is arranged to flare in an opposite orientation to the flared ends 144 of the stoppers, such that they can fit together to engage and keep the stoppers 140 in place. The connector 138 comprises a plurality of holes 150 and pegs 152, which are configured to engage with one another to connect the two halves of the connector block 138. The two halves are held together by the frictional connection between the holes 150 and pegs 152, which ensures the connector block 138 remains connected during normal use. The two halves can be detached by pulling them apart from one another. The two halves can be reattached and detached repeatedly as necessary. Preferably, each half comprises a combination of holes 150 and pegs 152, as this can improve the security of the connection.

The connector block 138 as described could be use in conjunction with magnet provided internally or externally to the head strap. If used with the second embodiment, in which the magnets are located on toggles slidable over the tubes, it is possible for the connector block 138 to be disconnected and removed, and the stoppers 140 and toggles 134 and 136 removed also. This can allow the straps to be removed completely and thoroughly cleaned and/or replaced.

It should be understood that the present invention has been described above purely by way of example, and modifications of detail can be made within the scope of the invention.

Each feature disclosed in the description, and (where appropriate) the claims and drawings may be provided independently or in any appropriate combination.

Reference numerals appearing in the claims are by way of illustration only and shall have no limiting effect on the scope of the claims.

### CONCLUSION

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. In the written description and claims, one or more steps within a method may be executed in a different order (or concurrently) without altering the principles of the present disclosure. Similarly, one or more instructions stored in a non-transitory computer-readable medium may be executed in different order (or concurrently) without altering the principles of the present disclosure. Unless indicated otherwise, numbering or other labeling of instructions or method steps is done for convenient reference, not to indicate a fixed order.

Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules, circuit elements, semiconductor layers, etc.) are described using various terms, including “connected,” “engaged,” “coupled,” “adjacent,” “next to,” “on top of,” “above,” “below,” and “disposed.” Unless explicitly described as being “direct,” when a relationship between first and second elements is described in the above disclosure, that relationship can be a direct relationship where no other intervening elements are present between the first and second elements, but can also be an indirect relationship where one or more intervening elements are present (either spatially or functionally) between the first and second elements.

The phrase “at least one of A, B, and C” should be construed to mean a logical (A OR B OR C), using a non-exclusive logical OR, and should not be construed to mean “at least one of A, at least one of B, and at least one of C.” The term “set” does not necessarily exclude the empty set—in other words, in some circumstances a “set” may have zero elements. The term “non-empty set” may be used to indicate exclusion of the empty set—in other words, a non-empty set will always have one or more elements. The term “subset” does not necessarily require a proper subset. In other words, a “subset” of a first set may be coextensive with (equal to) the first set. Further, the term “subset” does not necessarily exclude the empty set—in some circumstances a “subset” may have zero elements.

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, including the definitions below, the term “module” can be replaced with the term “controller” or the term “circuit.” In this application, the term “controller” can be replaced with the term “module.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); processor hardware (shared, dedicated, or group) that executes code; memory hardware (shared, dedicated, or group) that stores code executed by the processor hardware; other suitable hardware components that provide the described functionality; or a combination of some or all of the above, such as in a system-on-chip.

The module may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2020 (also known as the WWI wireless networking standard) and IEEE Standard 802.3-2018 (also known as the ETHERNET wired networking standard). Examples of a WPAN are IEEE Standard 802.15.4 (including the ZIGBEE standard from the ZigBee Alliance) and, from the Bluetooth Special Interest Group (SIG), the BLUETOOTH wireless networking standard (including Core Specification versions 3.0, 4.0, 4.1, 4.2, 5.0, and 5.1 from the Bluetooth SIG).

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module. For example, the client module may include a native or web application executing on a client device and in network communication with the server module.

Some or all hardware features of a module may be defined using a language for hardware description, such as IEEE Standard 1364-2005 (commonly called “Verilog”) and IEEE Standard 1076-2008 (commonly called “VHDL”). The hardware description language may be used to manufacture and/or program a hardware circuit. In some implementations, some or all features of a module may be defined by a language, such as IEEE 1666-2005 (commonly called “SystemC”), that encompasses both code, as described below, and hardware description.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

The memory hardware may also store data together with or separate from the code. Shared memory hardware encompasses a single memory device that stores some or all code from multiple modules. One example of shared memory hardware may be level 1 cache on or near a microprocessor die, which may store code from multiple modules. Another example of shared memory hardware may be persistent storage, such as a solid state drive (SSD), which may store code from multiple modules. Group memory hardware encompasses a memory device that, in combination with other memory devices, stores some or all code from one or more modules. One example of group memory hardware is a storage area network (SAN), which may store code of a particular module across multiple physical devices. Another example of group memory hardware is random access memory of each of a set of servers that, in combination, store code of a particular module.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of a non-transitory computer-readable medium are nonvolatile memory devices (such as a flash memory device, an erasable programmable read-only memory device, or a mask read-only memory device), volatile memory devices (such as a static random access memory device or a dynamic random access memory device), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. Such apparatuses and methods may be described as computerized apparatuses and computerized methods. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

What is claimed is:

1. A head strap for medical loupe glasses, comprising two tubes of flexible wipe-clean material and a magnet located on each tube, the magnets being connectable by magnetic forces to effect securement.

2. The head strap of claim 1, wherein the head strap has a smooth outer surface, preferably wherein the flexible wipe-clean material is silicone.

3. The head strap of claim 1, wherein the magnets are moveable.

4. The head strap of claim 3 wherein the magnets are located on toggles slidable over the tubes.

5. The head strap of claim 1, further comprising a connector block for connecting the two tubes, preferably wherein the weight of the connector block is adjustable.

6. The head strap of claim 5, wherein the connector block comprises two connectable parts, preferably wherein the two parts are removably connectable.

7. The head strap of claim 6, wherein the two parts comprise corresponding pegs and holes to facilitate connection.

8. The head strap of claim 6, further comprising a stopper on the end of each tube, configured to anchor the tubes in the connector block.

9. The head strap of claim 8, wherein the toggles, connector block and stoppers can be disconnected from the tubes and subsequently reconnected.

10. The head strap of claim 8, wherein the stoppers have flared ends.

11. The head strap of claim 10, wherein the connector block comprises cavities configured to accommodate and anchor the flared ends of the stoppers.

12. The head strap of claim 1, wherein the magnets have flat faces, preferably wherein the magnets have disc-shaped faces.

13. The head strap of claim 4, wherein the magnets are located on an external face of the toggles, that face being parallel to the tube.

14. The head strap of claim 4, wherein each toggle comprises a hole configured to accommodate the tube, and a dimension of the toggle parallel to the tube is reduced in the region of the hole.

15. The head strap of claim 1, wherein the tubes are configured to attach to temple arms of loupes and comprise a textured internal surface to engage with temple arms of loupes.

16. A loupe system comprising:
a loupe; and
a head strap, comprising two tubes of flexible wipe-clean material and a magnet located on each tube, the magnets being connectable by magnetic forces to effect securement.

17. The loupe system of claim 16, wherein the magnets are located on toggles slidable over the tubes.

18. The loupe system of claim 16, wherein the loupe comprises temple arms having curved and broadening ends, and the head strap further comprises a textured internal surface configured to engage the temple arms.

19. The loupe system of claim 16, wherein the loupe comprises one or more magnification lenses, and the head strap further comprises a connector block, wherein the weight of the connector block is configured to counterbalance the one or more magnification lenses.

20. The loupe system of claim 16, wherein the loupe comprises one or more magnification lenses and a head light, and the head strap further comprises a connector block, wherein the weight of the connector block is configured to counterbalance the combined weight of the one or more magnification lenses and the head light.
